(19) United States
(12) Patent Application Publication
(10) Pub. No.: US 2023/0331954 A1
(43) Pub. Date: Oct. 19, 2023

(54) MODIFIED COMPOUND AND MANUFACTURE METHOD THEREOF

(71) Applicant: National Yang Ming Chiao Tung University, Hsinchu City (TW)

(72) Inventors: Hsin-Chieh Lin, Hsinchu City (TW); Rajan Deepan Chakravarthy, Hsinchu City (TW); Satish Kumar Talloj, Hsinchu City (TW)

(73) Assignee: National Yang Ming Chiao Tung University, Hsinchu City (TW)

(21) Appl. No.: 18/119,446

(22) Filed: Mar. 9, 2023

Related U.S. Application Data
(60) Provisional application No. 63/269,111, filed on Mar. 10, 2022.

(51) Int. Cl. C08K 5/053; C07D 233/64

(52) U.S. Cl. CPC C08K 5/053; C07D 233/64

(57) ABSTRACT

In some embodiments of the present disclosure, a modified compound is provided, which includes a polyether segment having an end including oxygen, sulfur or nitrogen; an amino acid moiety; and a group, which is an acryl group or an alkylacryl group, in which the amino acid moiety is bonded to the end of the polyether segment through a carbonyl group, and the amino acid moiety is bonded to the group through an amino group. In some embodiments of the present disclosure, a method for manufacturing the modified compound is also provided.

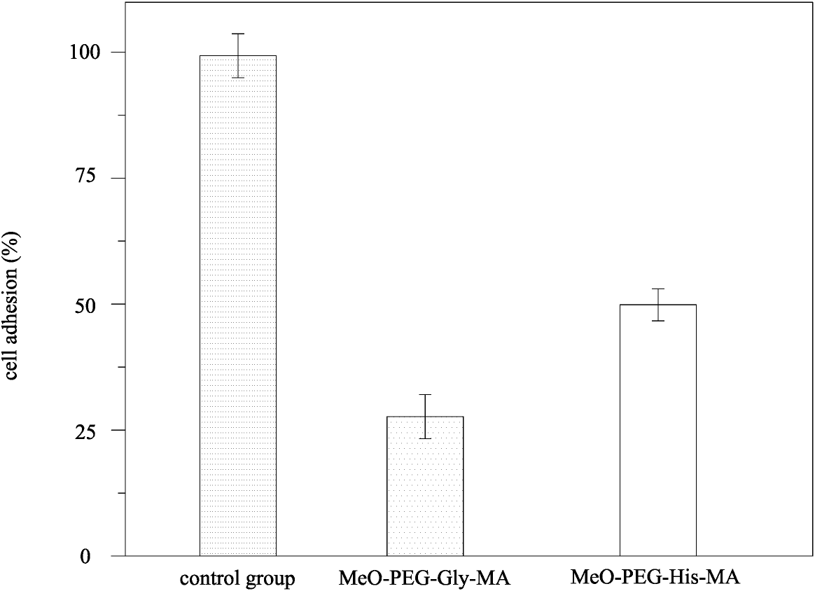

### CROSS - REFERENCE TO RELATED APPLICATION

This application claims priority to U.S. Provisional Application Serial Number 63/269,111, filed Mar. 10, 2022, which is herein incorporated by reference in its entirety.

### BACKGROUND

### Field of Invention

The present disclosure relates to a modified compound including a polyether segment and a manufacture method thereof.

### Description of Related Art

Polyether or polyether-modified compounds (connecting polyether with another functional group) are often used as materials for tissue engineering, such as repairing or replacing tissues. However, the existing polyether modified compounds are mainly modified to increase adhesion of cells, but those cannot solve an issue of adhesion in tissue engineering (e.g., adhesion to internal organs of the human body).

Therefore, how to improve an anti-adhesion effect of the modified compound is an issue to be solved.

### SUMMARY

In some embodiments of the present disclosure, a modified compound is provided, which includes a polyether segment having an end including oxygen, sulfur or nitrogen; an amino acid moiety; and a group, which is an acryl group or an alkylacryl group, in which the amino acid moiety is bonded to the end through a carbonyl group, and the amino acid moiety is bonded to the group through an amino group.

In some embodiments, the polyether segment includes a poly(ethylene glycol) segment, a polyalkylene glycol segment (e.g., polyvinyl alcohol (PVA) segment), a poly(propylene glycol) segment, a polyester polyol segment, a polyphenylene oxide segment, a poly[ethylene vinyl-co-alcohol] segment, a polysaccharide segment, or combinations thereof.

In some embodiments, the modified compound has a structure of following formula 1:

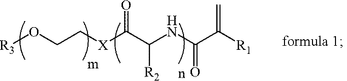

in which R₁ is hydrogen or an alkyl group including a carbon number of 1 to 20; R₂ is hydrogen, an alkyl group including a carbon number of 1 to 20, an amino acid side chain group or (CH₂)ₙNR′₃⁺; R₃ is hydrogen, an alkyl group including a carbon number of 1 to 20, (CH₂)ₙNR′₃⁺, (CH₂)ₙSO₃⁻, (CH₂)ₙCO₂⁻ or (CH₂)ₙPO₄⁻, X is oxygen, sulfur, or NR′; n is an integer from 1 to 20; m is an integer from 2 to 20,000; and R′ is hydrogen or an alkyl group including a carbon number of 1 to 20.

In some embodiments, the modified compound has a structure of following formula 2:

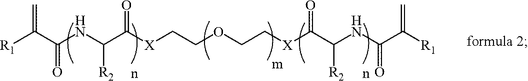

in which R₁ is hydrogen or an alkyl group including a carbon number of 1 to 20; R₂ is hydrogen, an alkyl group including a carbon number of 1 to 20, an amino acid side chain group or (CH₂)ₙNR′₃⁺; X is oxygen, sulfur or NR′; n is an integer from 1 to 20; m is an integer from 2 to 20,000; and R′ is hydrogen or an alkyl group including a carbon number of 1 to 20.

In some embodiments of the present disclosure, a method for manufacturing a modified compound is provided, which includes: mixing a first material having a polyether segment, a second material having a carboxyl group, and a solvent, in which the first material has an end group, and the end group is a hydroxyl group, a mercapto group, an amino group or a carboxyl group, and the second material is formed by reacting amino acid or peptide with alkacrylic acid, acrylic acid, or acyl halide having an acryl group or an alkylacryl group, and the second material reacts with the end group of the first material through a carboxyl group to obtain the modified compound.

In some embodiments, the step of mixing the first material, the second material, and the solvent is carried out in an inert gas environment.

In some embodiments, the first material is polyether, and the second material is N-methacryloylglycine.

In some embodiments, the polyether is polyethylene glycol monomethyl ether,

In some embodiments, the first material is polyether, and the second material is formed by reacting the peptide with the acyl halide having a methacryl group.

In some embodiments, the polyether is poly(ethylene glycol).

In some embodiments, the first material is polyether, and the second material is formed by reacting glycine with methacrylic acid or the acyl halide having a methacryl group.

In some embodiments, the polyether is poly(ethylene glycol).

In some embodiments of the present disclosure, a method for manufacturing a modified compound is provided, which includes: mixing a material having a polyether segment, an amino acid derivative having a protecting group, a solvent, and acyl halide having an acryl group or an alkylacryl group, in which the material has an end group, and the end group is a hydroxyl group, a mercapto group, an amino group or a carboxyl group, and the amino acid derivative reacts with the end group of the material through a carboxyl group, and the amino acid derivative reacts with the acyl halide through an amino group, to obtain the modified compound.

In some embodiments, the step of mixing the material having the polyether segment, the amino acid derivative having the protecting group, the solvent and the acyl halide includes: mixing the material, the amino acid derivative, and the solvent, allowing the amino acid derivative to react with the end group of the material through a carboxyl group to form a transition product, in which the transition product has an amino group; and mixing the transition product, the acyl halide, and the solvent, allowing the amino group of the transition product to react with the acyl halide to obtain the modified compound.

In some embodiments, the material is polyether, and the amino acid derivative is histidine derivative, and the acyl halide is methacryl halide.

In some embodiments, the material is polyether, and the amino acid derivative is histidine derivative, and the acyl halide is methacryl halide.

### BRIEF DESCRIPTION OF THE DRAWINGS

In order to allow the above-mentioned and other purposes, features, advantages and embodiments of the present disclosure to be more clearly understood, accompanying drawing is described as follows:

This FIGURE illustrates a graph of results of a bioadhesion test performed on modified compounds according to one embodiment of the present disclosure.

### DETAILED DESCRIPTION

In order that the present disclosure is described in detail and completeness, implementation aspects and specific embodiments of the present disclosure with illustrative description are presented; but those are not the only form for implementation or use of the specific embodiments of the present disclosure. The embodiments disclosed herein may be combined or substituted with each other in an advantageous manner, and other embodiments may be added to an embodiment without further description. In the following description, numerous specific details will be described in detail in order to enable the reader to fully understand the following embodiments. However, the embodiments of the present disclosure may be practiced without these specific details.

In this description, unless the context specifically dictates otherwise, “a” and “the” may mean a single or a plurality. It will be further understood that “comprise”, “include”, “have”, and similar terms as used herein indicate described features, regions, integers, steps, operations, elements and/or components, but not exclude other features, regions, integers, steps, operations, elements, components and/or groups.

In this description, “derivative” means a product derived from a compound that an atom or an atomic group therein is directly or indirectly replaced by another atom or atomic group.

Although a series of operations or steps are described below to illustrate the method disclosed herein, the order of the operations or steps is not to be construed as limiting. For example, some operations or steps may be performed in a different order and/or concurrently with other steps. In addition, not all illustrated operations, steps, and/or features must be performed to implement embodiments of the present disclosure. Moreover, each of the operations or steps described herein may include a plurality of sub-steps or actions.

An aspect of the present disclosure provides a modified compound including a polyether segment having an end including oxygen, sulfur or nitrogen; an amino acid moiety; and a group, which is an acryl group or an alkylacryl group, in which the amino acid moiety is bonded to the end of the polyether segment through a carbonyl group, and the amino acid moiety is bonded to the group through an amino group.

In some embodiments, the modified compound has a structure of following formula 1 (or referred to as a first modified compound):

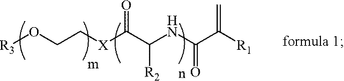

in which R₁ is hydrogen or an alkyl group including a carbon number of 1 to 20 (e.g., the carbon number is 1, 2, 3, 4, 5, 6, 7, 8, 9, 10, 11, 12, 13, 14, 15, 16, 17, 18, 19, 20 or a value in any of the aforementioned intervals), R₂ is hydrogen, an alkyl group including a carbon number of 1 to 20, (e.g., the carbon number is 1, 2, 3, 4, 5, 6, 7, 8, 9, 10, 11, 12, 13, 14, 15, 16, 17, 18, 19, 20 or a value in any of the aforementioned intervals), an amino acid side chain group or (CH₂)ₙNR′₃⁺; R₃ is hydrogen, an alkyl group including a carbon number of 1 to 20, (e.g., the carbon number is 1, 2, 3, 4, 5, 6, 7, 8, 9, 10, 11, 12, 13, 14, 15, 16, 17, 18, 19, 20 or a value in any of the aforementioned intervals), (CH₂)ₙNR′₃⁺, (CH₂)ₙSO₃⁻, (CH₂)ₙCO₂⁻ or (CH₂)ₙPO₄⁻; X is oxygen, sulfur, or NR′; n is an integer from 1 to 20 (e.g., 5, 10, 15, 20 or a value in any of the aforementioned intervals); m is an integer greater than or equal to 2 (e.g., 2 to 20,000, such as 2, 5, 10, 20, 30, 40, 50, 60, 70, 80, 90, 100, 150, 200, 500, 1,000, 2,000, 3,000, 4,000, 5,000, 6,000, 7,000, 8,000, 9,000, 10,000, 15,000, 20,000 or a value in any of the aforementioned intervals); and R′ is hydrogen or an alkyl group including a carbon number of 1 to 20 (e.g., the carbon number is 1, 2, 3, 4, 5, 6, 7, 8, 9, 10, 11, 12, 13, 14, 15, 16, 17, 18, 19, 20 or a value in any of the aforementioned intervals).

In some embodiments, a molecular weight of the first modified compound is 200 Da (Dalton) to 60000 Da, such as 200 Da, 500 Da, 1,000 Da, 2,500 Da, 5,000 Da, 7,500 Da, 10,000 Da, 20,000 Da, 30,000 Da, 40,000 Da, 50,000 Da, 60,000 Da or a value in any of the aforementioned intervals.

In some embodiments, R₂ is selected from the group consisting of amino acid side chain groups of glycine (Gly), alanine (Ala), valine (Val), serine (Ser), phenylalanine (Phe), lysine (Lys), threonine (Thr), methionine (Met), tyrosine (Tyr), histidine (His), aspartic acid (Asp), glutamic acid (Glu), asparagine (Asn), glutamine (Gln), cysteine (Cys), selenocysteine (Sec), isoleucine (Ile), leucine (Leu), arginine (Arg) and tryptophan (Trp).

In one embodiment, the modified compound is methyl ether-poly(ethylene glycol)-glycine-methyl acrylate (MeO-PEG-Gly-MA, the first modified compound A), in which in formula 1, R₁ is a methyl group, R₂ is a side chain group of Gly, R₃ is a methyl group, and X is oxygen. In another embodiment, the modified compound is methyl ether-poly(ethylene glycol)-histidine-methyl acrylate (MeO-PEG-His-MA, the first modified compound B), in which in formula 1, R₁ is a methyl group, R₂ is a side chain group of His, R₃, is a methyl group, and X is oxygen. The first modified compound B is basically similar to the first modified compound A, and the difference therebetween is that R₂ thereof are different.

In one embodiment, the modified compound is methyl ether-poly(ethylene glycol)-triglycine-methyl acrylate (MeO-PEG-(Gly)₃-MA, the first modified compound C), in which in formula 1, R₁ is a methyl group, R₂ is a side chain group of Gly, R₃ is hydrogen, and X is oxygen. In another embodiment, the modified compound is methyl ether-poly(ethylene glycol)-trialanine-methyl acrylate (MeO-PEG-(Ala)₃-MA, the first modified compound D), in which in formula 1, R₁ is a methyl group, R₂ is a side chain group of Ala, R₃ is hydrogen, and X is oxygen. The first modified compound C is basically similar to the first modified compound D, and the difference therebetween is that R₂ thereof are different.

It is worth emphasizing that the polyether segment has —CH₂CH₂O—repeating units, and these repeating units can generate hydrogen bonds with water, thereby forming a hydration layer on a surface to avoid protein sticking. Further, the first modified compound (e.g., the first modified compound A, B, C or D) is designed that the acryl group or the alkylacryl group (the aforementioned group) and the peptide bond are located on the same side of the polyether segment, so that the peptide bond between the first modified compound makes the group close to the polyether segment due to the hydrogen bond effect to change orientation of the polyether segment. Compared with unmodified polyethylene glycol monomethyl ether, or another compound that the group and the peptide bond are located on two different sides of the polyether segment, the first modified compound can have better anti-bioadhesion effect through the aforementioned structural properties. In one embodiment, Presto Blue Cell Viability is used to test adsorption effect of polyethylene glycol monomethyl ether (control group), the first modified compound A (MeO-PEG-Gly-MA) or the first modified compound B (MeO-PEG-His-MA) to mouse fibroblast L929, please refer to the FIGURE for results. The FIGURE shows that compared with the unmodified control group (polyethylene glycol monomethyl ether), the modified first modified compound (the first modified compound A (MeO-PEG-Gly-MA) or the first modified compound B (MeO-PEG-His-MA)) can reduce cell adhesion.

ln some embodiments, the modified compound has a structure of following formula 2 (or referred to as a second modified compound):

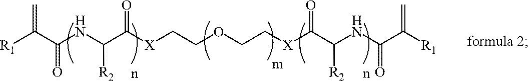

in which R₁ is hydrogen or an alkyl group including a carbon number of 1 to 20 (e.g., the carbon number is 1, 2, 3, 4, 5, 6, 7, 8, 9, 10, 11, 12, 13, 14, 15, 16, 17, 18, 19, 20 or a value in any of the aforementioned intervals); R₂ is hydrogen, an alkyl group including a carbon number of 1 to 20 (e.g., the carbon number is 1, 2, 3, 4, 5, 6, 7, 8, 9, 10, 11, 12, 13, 14, 15, 16, 17, 18, 19, 20 or a value in any of the aforementioned intervals), an amino acid side chain group or (CH₂)ₙNR′₃⁺; X is oxygen, sulfur or NR′; n is an integer from 1 to 20 (e.g., 5, 10, 15, 20 or a value in any of the aforementioned intervals); m is an integer greater than or equal to 2 (e.g., 2 to 20,000, such as 2, 5, 10, 20, 30, 40, 50, 60, 70, 80,90, 100, 150, 200, 500, 1,000, 2,000, 3,000, 4,000, 5,000, 6,000, 7,000, 8,000 9,000, 10,000, 15,000, 20,000 or a value in any of the aforementioned intervals); and R′ is hydrogen or an alkyl group including a carbon number of 1 to 20 (e.g., the carbon number is 1, 2, 3, 4, 5, 6, 7, 8, 9, 10, 11, 12, 13, 14, 15, 16, 17, 18, 19, 20 or a value in any of the aforementioned intervals).

In some embodiments, R₂ is selected from the group consisting of amino acid side chain groups of glycine (Gly), alanine (Ala), valine (Val), serine (Ser), phenylalanine (Phe), lysine (Lys), threonine (Thr), methionine (Met), tyrosine (Tyr), histidine (His), aspartic acid (Asp)), glutamic acid (Glu), asparagine (Asn), glutamine (Gln), cysteine (Cys), selenocysteine (Sec), isoleucine (Ile), leucine (Leu), arginine (Arg) and tryptophan (Trp). In one embodiment, the second modified compound is methyl acrylate-glycine-poly(ethylene glycol)-glycine-methyl acrylate (MA-Gly-PEG-Gly-MA), and R₁ is a methyl group, and R₂ is a side chain group of Gly or Ala.

It can be understood that, relative to unmodified polyethylene glycol monomethyl ether, or another compound including a polyether segment that the acryl group or the alkylacryl group (i.e., the aforementioned group) and the peptide bond are located on two different sides of the polyether segment, the second modified compound can have better anti-bioadhesion effect through the design that the group and the peptide bond are located on the same side of the polyether segment to regulate orientation of the polyether segment.

Another aspect of the present disclosure provides a method for manufacturing a modified compound, including: mixing a first material having a polyether segment, a second material having a carboxyl group and a peptide bond, and a solvent, in which the first material has an end group, and the end group is a hydroxyl group, a mercapto group, an amino group, or a carboxyl group, and the second material is formed by reacting amino acid or peptide with alkacrylic acid, acrylic acid, or acyl halide having an acryl group or an alkylacryl group, and the second material reacts with the end group of the first material through a carboxyl group to obtain the first modified compound.

In some embodiments, the solvent may be an organic solvent. The organic solvents include, for example, alcohols, ketones, chloroform, glycerides, aromatic hydrocarbons, or combinations thereof, such as dichloromethane (abbreviated as DCM), chloroform, ethanol, acetonitrile, toluene, acetone, hexane, or combinations thereof.

In some embodiments, in the step of mixing the first material having the polyether segment, the second material having the carboxyl group and the solvent, the first material is polyethylene glycol monomethyl ether, and the second material is N-methacryloylglycine, and the solvent is dichloromethane, and thus the first modified compound A (MeO-PEG-Gly-MA) having aforementioned formula 1 is generated.

In some embodiments, in the step of mixing the first material having the polyether segment, the second material having the carboxyl group and the solvent, the first material is polyether (e.g., poly(ethylene glycol)), and the second material is formed by reacting peptide with acyl halide having a methacryl group, and the solvent is dichloromethane. In one embodiment, the first material is polyether (e.g., poly(ethylene glycol)), and the second material is N-methacryloyl triglycine formed by reacting triglycine acid with methacryloylchloride, and the solvent is dichloromethane, and thus the first modified compound C (MeO-PEG-3Gly-MA) having aforementioned formula 1 is generated. In other embodiments, the first modified compound C is subjected to an esterification reaction to further generate the first modified compound D (MeO-PEG-3Ala-MA).

In some embodiments, in the step of mixing the first material having the polyether segment, the second material having the carboxyl group and the solvent, the first material is polyether (e.g., poly(ethylene glycol)), and the second material is N-methacryloylglycine formed by reacting glycine with methacrylic acid or methacryloylchloride, and the solvent is dichloromethane, and thus the second modified compound (MA-Gly-PEG-Gly-MA) having aforementioned formula 2 is generated.

In some embodiments, the step of mixing the first material, the second material, and the solvent includes stirring the first material, the second material and the solvent at a temperature in a range of from 20° C. to 40° C. (e.g., 20° C., 25° C., 30° C., 35° C., 40° C. or a value in any of the aforementioned intervals) for 8 hours to 120 hours (e.g., 8 hours, 10 hours, 12 hours, 14 hours, 16 hours, 24 hours, 36 hours, 48 hours, 60 hours, 72 hours, 84 hours, 96 hours, 108 hours, 120 hours, or a value in any of the aforementioned intervals). If the temperature is too low or too high, or the time is too long or too short, the yield will be affected. In some embodiments, the step of mixing the first material, the second material and the solvent is performed in an inert gas (e.g., nitrogen) environment to prevent the first material or the second material from reacting with components in the air. In some embodiments, in the step of mixing the first material, the second material, and the solvent, a weight ratio of the first material to the second material is in a range of from 1:1 to :8:1, such as 1:1, 1.075: 1, 1.35 :1, 1.65:1, 1.84:1, 2:1, 2.075:1, 2,35:1, 2.65:1, 2.84:1, 3:1, 3.075:1, 3,35:1, 3.65:1, 3.84:1, 4:1, 4.075:1, 4,35:1, 4.65:1, 4.84:1, 5:1, 5.075:1, 5,35:1, 5,65:1, 5.84:1, 6:1, 8.075:1, 6,35:1, 6,65:1, 6.84:1, 7:1, 7.075:1, 7,35:1, 7,65:1, 7.84:1, 8:1, or a value in any of the aforementioned intervals.

In one embodiment, a method for manufacturing the aforementioned first modified compound A may be represented by following reaction formula.

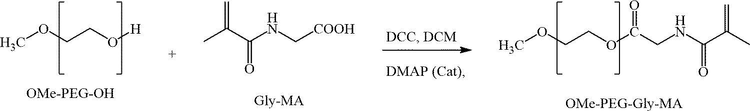

As shown in the above reaction formula, in a nitrogen environment, N-methacryloylglycine (abbreviated as Gly-MA, 0.4 g, 2.8 mmol; the second material) and 4-(dimethylamino)pyridine (abbreviated as DMAP, 30 mg, 0.25 mmol) were added. Next, dry dichloromethane (DCM, 25 ml; the organic solvent) was added, and mixed and stirred for 15 minutes. Next, N,N′-dicyclohexylcarbodiimide (abbreviated as DCC, 0.576 g, 2.8 mmol) was added under ice-cooling condition, and stirred for 15 minutes. Polyethylene glycol monomethyl ether (MeO-PEG-OH, the molecular weight was 2,000, 4.6 g; the first material) was added, and warmed to room temperature, and stirred overnight. Next, a solid precipitate of N,N′-dicyclohexylurea was removed by filtration, and the filtrate was concentrated under reduced pressure and then purified using cold diethyl ether to obtain the first modified compound A of colorless solid (3.3 g). Property measurement results of the first modified compound A were as follows. ¹H NMR (500 MHz, CDCl₃) δ 5.76 (s, 1H), 5.37 (s, 1H), 4.32-4.30 (m, 2H), 4.12-4.11 (m, 2H) 3.65-3.54 (m, 190 H), 3.36 (s, 3H), 1.97 (s, 3H).

In one embodiment, a method for manufacturing the aforementioned first modified compound C may be represented by following Reaction formula 1 and Reaction formula 2.

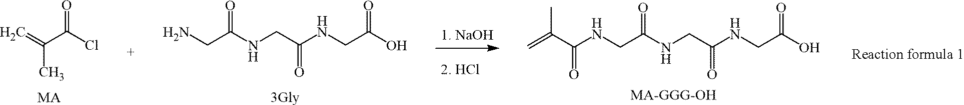

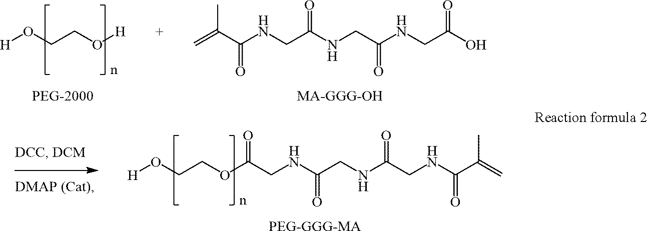

First, as shown in above Reaction formula 1, N-methacryloyl triglycine (MA-GGG-OH, or (Gly)₃-MA) was prepared. Specifically, in a nitrogen environment, triglycine ((Gly)₃, 1 g, 5.3 mmol) was added to a solvent including dichloromethane (40 ml) and aqueous sodium hydroxide (6 N, 1.3 ml) to obtain a triglycine solution. Next, under ice-cooling condition, methacryloylchloride (0.831 g, 7.95 mmol) dissolved in 20 ml of DCM was slowly dropped into the triglycine solution over a period of 2 hours. Next, the pH value was adjusted to 9-10.5 using sodium hydroxide and then stirred for 30 minutes. After the reaction was completed, an organic phase was separated, and an aqueous phase was acidified using 1N hydrogen chloride to pH value of 1.5 and then stood to obtain a solid substance, which was N-methacryloyl triglycine ((Gly)₃-MA, and the yield was close to 90%), and property measurement results of (Gly)₃-MA were as follows. ¹H NMR (600 MHz, DMSO) δ 8.20 (m, 3H), 5.75 (s, 1H), 5.42-5.33 (m, 1H), 3.80-3.69 (m, 6H), 1.89 (d, J=19.3 Hz, 3H).

Next, as shown in above Reaction formula 2, in a nitrogen environment, (Gly)₃-MA (0.424 g, 1.6 mmol; the second material) and 4-(dimethylamino)pyridine (DMAP, 30 mg, 0.25 mmol) were added. Next, dry dichloromethane (DCM, 25 ml; the organic solvent) was added, and mixed and stirred for 15 minutes. Next, N,N′-dicyclohexylcarbodiimide (DCC, 0.340 g, 1.6 mmol) was added under ice-cooling condition, and stirred for 15 minutes. Polyethylene glycol (PEG, the molecular weight was 2,000, 3 g; the first material) was added, and warmed to room temperature, and stirred for 4 days. Next, a solid precipitate of N,N′-dicyclohexylurea was removed by filtration, and the filtrate was concentrated under reduced pressure, and then the DCM suspension filtrate was used as a DCM mixed liquid. The aqueous phase of the DCM mixed liquid was very viscous, and a colloidal substance was observed between the aqueous phase and the organic phase. Next, the organic phase of the DCM mixed liquid was separated, and after the organic phase was dried using magnesium sulfate, the first modified compound C of colorless solid was obtained. Due to limited solubility of (Gly)₃-MA in DCM, binding efficiency of (Gly)₃-MA to PEG was limited. Therefore, although the yield of (Gly)₃-MA reached about 90%, the yield of the first modified compound C was only 70%. Property measurement results of the first modified compound C were as follows. ¹H NMR (500 MHz, DMSO) δ 5.81 (s, 1H), 5.41-5.34 (m, 1H), 4.30-4.23 (m, 2H), 4.08-3.97 (m, 6H), 3.79-3.45 (m, 268 H), 3.36 (s, 4.2H), 1.97 (s, 3H).

In another embodiment, the first modified compound C may be dissolved in an organic solvent (e.g., dimethylformamide; DMF), and the first modified compound C is further esterified into the first modified compound D. That is, the side chain group of Gly (hydrogen) is esterified to the methyl group (the side chain group of Ala).

In one embodiment, a method for manufacturing the aforementioned second modified compound may be represented by following reaction formula.

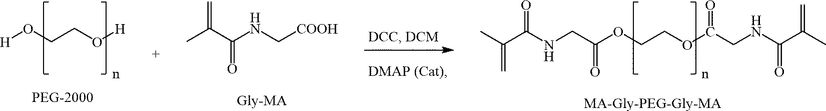

As shown in the above reaction formula, in a nitrogen environment, N-methacryloylglycine (Gly-MA, 1.37 g, 9.6 mmole; the second material) and 4-(dimethylamino)pyridine (DMAP, 30 mg, 0.25 mmol) were added. Next, dry dichloromethane (DCM, 25 ml; the organic solvent) was added, and mixed and stirred for 15 minutes. Next, N,N′-dicyclohexylcarbodiimide (DCC, 1.65 g, 8 mmol) was added under ice-cooling condition, and stirred for 15 minutes. Polyethylene glycol (PEG, the molecular weight was 2,000, 8 g; the first material), and warmed to room temperature, and stirred overnight. Next, a solid precipitate of N,N′-dicyclohexylurea was removed by filtration, and a filtrate was concentrated under reduced pressure, and then the DCM suspension filtrate was used as a DCM mixed liquid. The aqueous phase of the DCM mixed liquid was very viscous, and a colloidal substance was observed between the aqueous phase and the organic phase. Next, the organic phase was separated, and after the organic phase was dried using magnesium sulfate, the second modified compound of colorless solid (yield of 70%) was obtained. Property measurement results of the second modified compound were as follows. ¹H NMR (600 MHz, CDCl₃) δ 6.44 (bs, 2H), 5.76 (s, 2H), 5.37 (d, J=0.9 Hz, 2H), 4.33-4.28 (m, 4H), 4.11 (d, J=5.2 Hz, 4H), 3.79-3.47 (m, 198H), 1.97 (d, J=0.7 Hz, 6H).

Another aspect of the present disclosure provides a method for manufacturing a modified compound, which includes mixing a material having a polyether segment, an amino acid derivative having a protecting group, a solvent, and acyl halide having an acryl group or an alkylacryl group, in which the material has an end group, and the end group is a hydroxyl group, a mercapto group, an amino group or a carboxyl group, and the amino acid derivative reacts with the end group of the material through a carboxyl group, and the amino acid derivative reacts with the acyl halide through an amino group, to obtain the modified compound.

In some embodiments, the step of mixing the material having the polyether segment, the amino acid derivative having the protecting group, the solvent, and the acyl halide includes first stirring the material, the amino acid derivative and the solvent at a temperature in a range of from 20° C. to 40° C. (e.g., 20° C., 25° C., 30° C., 35° C., 40° C. or a value in any of the aforementioned intervals) until clear, and then cooling to -5° C. to 5° C. (e.g., -5° C., -4° C., -3° C., -2° C., -1℃, 0° C., 1° C., 2° C., 3° C., 4° C., 5° C. or a value in any of the aforementioned intervals), to increase a yield of a transition product. In some embodiments, the step of mixing the material having the polyether segment, the amino acid derivative, and the solvent is performed in an inert gas (e.g., nitrogen) environment to prevent the material or the amino acid derivative from reacting with components in the air. In some embodiments, in the step of mixing the material, the amino acid derivative and the solvent, a weight ratio of the material to the amino acid derivative is in a range of from 2:1 to 6:1, such as 2:1, 3.225:1, 4.225:1, 5.225:1, 6:1, or a value in any of the aforementioned intervals. If the weight ratio of is too high or too low, the yield of the transition product will be affected.

In some embodiments, the step of mixing the material, the amino acid derivative, and the solvent includes reacting at a temperature in a range of from -5° C. to 5° C. (e.g., -5° C., -4° C., -3° C., -2° C., -1° C., 0° C., 1° C., 2° C., 3° C., 4° C., 5° C. or a value in any of the aforementioned intervals) in the dark for 16 hours to 36 hours (e.g., 16 hours, 20 hours, 24 hours, 28 hours, 32 hours, 36 hours, or a value in any of the aforementioned intervals), to increase a yield of the modified compound. In some embodiments, the step of mixing the transition product, the acyl halide, and the solvent is performed in an inert gas (e.g., nitrogen) environment to prevent the transition product or the acyl halide from reacting with components in the air. In some embodiments, in the step of mixing the transition product, the acyl halide and the solvent, a weight ratio of the transition product to the acyl halide is in a range of from 2:1 to 10:1, such as 2:1, 3:1, 4:1, 5:1 1, 6:1, 7:1, 8:1, 9:1, 10:1 or a value in any of the aforementioned intervals. If the weight ratio is too high or too low, the yield of the modified compound will be affected.

In some embodiments, the material having the polyether segment is polyether (e.g., polyethylene glycol monomethyl ether), and the amino acid derivative is a histidine derivative having a protecting group (e.g., N-fluorenylmethoxycarbonyl-N′-trityl-L-histidine, in which the amino group is connected to the first protecting group (N-fluorenylmethoxycarbonyl), and N on the side chain group of histidine is connected to the second protecting group (trityl group) to prevent the amino group and N on the side chain group of histidine from reacting with other substances in advance), and the acyl halide is methacryl halide (e,g., methacryloylchloride), and the solvent is dichloromethane, and thus the first modified compound B (OMe-PEG-His-MA) having aforementioned formula 1 may be generated.

For example, a method for manufacturing the aforementioned first modified compound B may be represented by following reaction formula, which includes Step-1 to Step-4.

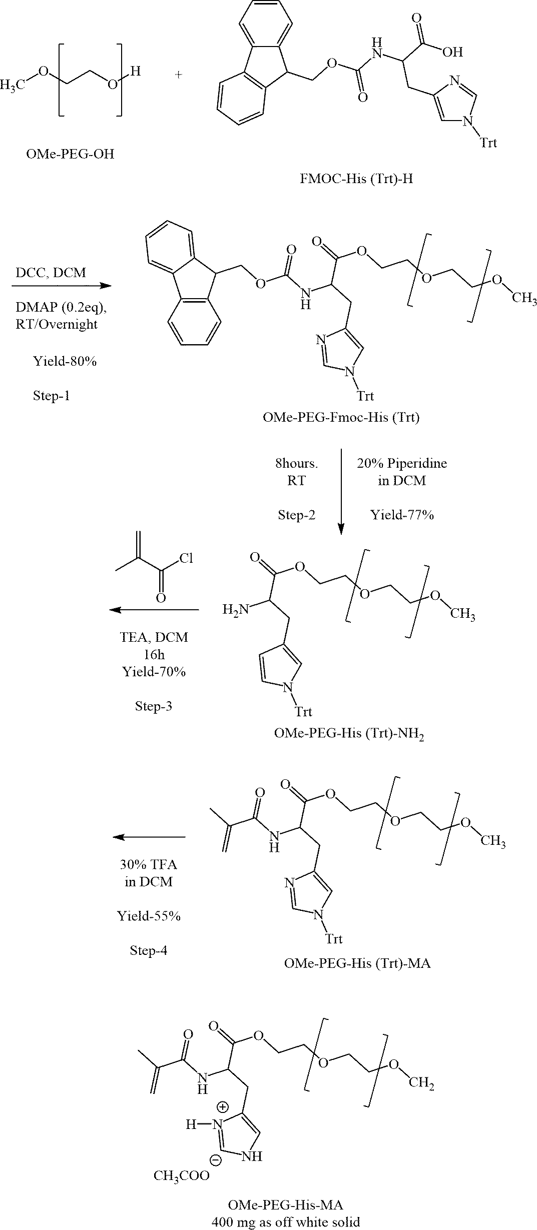

As shown in the above reaction formula, first, Step-1 is performed to obtain methyl ether-poly(ethylene glycol)-fluorenylmethoxycarbonyl-histidine(trityl) (MeO-PEG-Fmoc-His(Trt)).

Specifically, under a nitrogen atmosphere, N-fluorenylmethoxycarbonyl-N′-trityl-L-histidine (Fmoc-His (Trt)-OH, 2.0 g, 3.22 mmol; the amino acid derivative) was dissolved in anhydrous dichloromethane (DCM, 50 ml). Catalytic 4-(dimethylamino)pyridine (DMAP, 78.2 mg, 0.64 mmol) was added at room temperature. Next, at room temperature, polyethylene glycol monomethyl ether (MeO-PEG-OH, the molecular weight was 2,000, 6.45 g, 3.22 mmol; the material having the polyether segment) was added to make polyethylene glycol monomethyl ether dissolve completely. After the solution became clear, it was cooled to 0° C. Next, under an inert gas environment, N,N′-dicyclohexylcarbodiimide (DCC, 0.74 g, 3.55 mmol) was added in one go, and stirred at room temperature for 16 hours. At this time, dicyclohexylurea (DCU) was precipitated as white solid. The reaction liquid was filtered to remove DCU, and the filtrate was washed with a minimal amount of DCM. Next, the filtrate was washed using saturated saline solution extracted twice with DCM. The organic layer was dried using magnesium sulfate (MgSO₄), and filtered and evaporated under reduced pressure to obtain colorless oil. The oil was precipitated by adding cold diethyl ether and stirred for 10 minutes, and the precipitate was filtered to obtain the finished product of Step 1 (MeO-PEG-Fmoc-His(Trt), 6.82 g, yield of 80%) as white solid. Property measurement results of the finished product of Step 1 above were as follows. ¹H NMR(600 MHz, COCl₃): δ 8.16 (d, J=6 Hz, 1H), 7.74 (d, J=6 Hz, 2H), 7.61 (t, J =6 Hz, 2H), 7.39-7.36 (m, 3H), 7.32-7.31(m, 8H), 7.29-7.26 (m, 1H), 7.11-7.09 (m, 7H), 6.60 (s, 1H), 6.50 (m, 2H), 4.36 (m, 2H), 4.35-4.19 (m, 6H) 3.75-3.51 (m, 160H), 3.37 (s, 3H), 3.16 (s, 1H), 3.10-3.05 (m, 2H), which conformed to properties of MeO-PEG-Fmoc-His(Trt).

Next, Step-2 is performed to obtain methyl ether-poly(ethylene glycol)-histidine(trityl)-amine (MeO-PEG-His(Trt)-NH₂).

Specifically, first, MeO-PEG-Fmoc-His(Trt) (2.0 g, 0.76 mmol) was dissolved in anhydrous DCM (20 ml) and cooled to 10° C. Next, 2 ml of piperidine (dissolved in DCM, 20 wt%) was added dropwise to the stirring reaction solution, and the reaction solution was warmed to room temperature and stirred continuously for 6 hours to remove Fmoc. Through thin layer chromatography (TLC), under a washing condition of 20% ethyl acetate(EA)-n-hexane(Hex), there were highly non-polar spots with 9-dibenzofulvene by-product, and intensity remained constant after 6 hours, confirming successful removal of all Fmoc. Next, the reactant after removal of Fmoc was evaporated to dryness and rinsed with DCM to remove most of piperidine to obtain colorless oil. The colorless oil was precipitated using cold diethyl ether and stirred for 10 minutes, and then filtered and washed with excess diethyl ether to obtain a finished product of Step 2 (MeO-PEG-His(Trt)-NH₂, 1.4 g, yield of 77%; the transition product). Property measurement results of the finished product of Step 2 above were as follows. ¹H NMR (600 MHz, CDCl₃): δ 7.35 (s,1H), 7.33-7.31 (m,8H), 7.12-7.09 (m, 7H), 6.60 (s, 1H), 6.50 (d, 2H), 4.22-4.14 (m,2H), 3.82-3.80 (s,2H),3.75-3.52 (m, 140H), 3.37 (s, 3H), 3.16 (s, 1H), 3.01-2.99 (m, 2H), Fmoc signal was disappeared, and those conformed to properties of MeO-PEG-His(Trt)-NH₂.

Next, Step-3 is performed to obtain methyl ether-poly(ethylene glycol)-histidine(trityl)-methyl acrylate (MeO-PEG-His(Trt)-MA).

Specifically, first, MeO-PEG-His(Trt)-NH₂ (1.3 g, 0.54 mmol; the transition product) and excess triethylamine (300 µL, 2.19 mmol) were dissolved in dry DCM (20 ml) and cooled to 0° C. under nitrogen. Excess methacryloylchloride (214 µl, 2.19 mmol, 0.22893 g; the alkacrylic acid derivative) dissolved in 4 ml of DCM was added to the reaction solution at 0° C. The reaction solution was in the dark and reacted continuously at room temperature for 24 hours. An organic phase was separated by adding saturated saline solution and extracted twice with DCM. The organic phase was dried filtered using magnesium sulfate and rotary evaporated in the dark at 45° C. to obtain a viscous liquid. Cold diethyl ether was added to precipitate the viscous liquid, and then filtered to obtain a finished product of Step 3 (MeO-PEG-His(Trt)-MA, 1.02 g, yield of 70%) as pale yellow solid. Property measurement results of the finished product of Step 3 above were as follows. ¹HNMR (600 MHz, CDCl₃): δ 7.6 (m, 1H), 7.48-7.32 (m, 9H), 7.15-6.95 (m, 6H), 6.89 (m, 1H), 5.91 (s, 1H), 5.39 (s, 1H), 4.89-4.80 (m, 2H), 4.25 (bs, 3H), 3.65-3.55 (m, 163H), 3.37 (s, 3H), 3.12-3.08 (m, 2H), 1.96 (s, 3H), which conformed to properties of MeO-PEG-His(Trt)-MA.

Next, Step-4 is performed to obtain methyl ether-poly(ethylene glycol)-histidine-methyl acrylate (MeO-PEG-His-MA).

Specifically, first, the finished product MeO-PEG-His(Trt)-NH₂ (0.8 g, 0.32 mmol) of Step 3 in 10 ml of DCM was added in 20ml of 30% trifluoroacetic acid (TFA) (in which TFA was dissolved in DCM), and stirred at room temperature for 5 hours to form trityl carbocation, making the reaction solution appear thick yellow. After the solvent was removed by rotary evaporation, excess TFA was distilled off by rinsing twice with DCM to give yellow oil. The yellow oil was decolorized, and a target substance was precipitated using cold diethyl ether. The precipitate (target substance) was centrifuged and dried under vacuum for 2 hours to obtain a finished product of Step 4 (MeO-PEG-His-MA, 0.4 g, yield of 55%; the first modified compound B) as pale yellow solid. Property measurement results of the finished product of Step 4 above were as follows. ¹HNMR (600 MHz, CDCl₃): δ 8.63 (m, 2H), 7.25 (s, 1H), 5.85 (s, 1H), 5.40 (s, 1H), 4.81 (m, 2H), 4.50-4.44 (m, 2H), 4.14 (m, 2H), 3.65-3.55 (m, 163H), 3.37 (s, 3H), 3.12-3.08 (m, 2H), 1.96 (s, 3H), which conformed to properties of MeO-PEG-His-MA. The modified compounds provided by some embodiments of the present disclosure, compared with conventional polyethylene glycol monomethyl ether, or another compound that the acryl group or the alkylacryl group, and the peptide bond are located on two different sides of the polyethylene glycol segment, the modified compounds can have better anti-bioadhesion effect through the design that the acryl group or the alkylacryl group, and the peptide bond are located on the same side of the polyether segment to change orientation of the polyether segment

Although the disclosure has been disclosed in the above embodiments, it is not intended to limit the disclosure, and it is to be understood that those skilled in the art can make various changes and modifications without departing from the spirit and scope of the disclosure. The scope of protection of the present disclosure is subject to the definition of the scope of claims.

What is claimed is:

1. A modified compound, comprising:
a polyether segment having an end comprising oxygen, sulfur, or nitrogen;
an amino acid moiety; and
a group being an acryl group or an alkylacryl group,
wherein the amino acid moiety is bonded to the end of the polyether segment through a carbonyl group, and the amino acid moiety is bonded to the group through an amino group.

2. The modified compound of claim 1, wherein the polyether segment comprises a poly(ethylene glycol) segment, a polyalkylene glycol segment, a poly(propylene glycol) segment, a polyester polyol segment, a polyphenylene oxide segment, a poly[ethylene vinyl-co-alcohol] segment, a polysaccharide segment, or combinations thereof.

3. The modified compound of claim 1, wherein the modified compound has a structure of following formula 1: 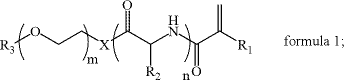
wherein R₁ is hydrogen or an alkyl group including a carbon number of 1 to 20;
R₂ is hydrogen, an alkyl group including a carbon number of 1 to 20, an amino acid side chain group, or (CH₂)ₙNR′₃⁺;
R₃ is hydrogen, an alkyl group including a carbon number of 1 to 20, (CH₂)ₙNR′₃⁺, (CH₂)ₙSO₃⁻, (CH₂)ₙCO₂⁻ or (CH₂)ₙPO₄⁻;
X is oxygen, sulfur, or NR′;
n is an integer from 1 to 20;
m is an integer from 2 to 20,000; and
R′ is hydrogen or an alkyl group including a carbon number of 1 to 20.

4. The modified compound of claim 1, wherein the modified compound has a structure of following formula 2: 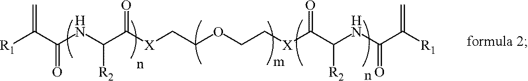
wherein R₁ is hydrogen or an alkyl group including a carbon number of 1 to 20;
R₂ is hydrogen, an alkyl group including a carbon number of 1 to 20, an amino acid side chain group or (CH₂)ₙNR′₃⁺;
X is oxygen, sulfur or NR′;
n is an integer from 1 to 20;
m is an integer from 2 to 20,000; and
R′ is hydrogen or an alkyl group including a carbon number of 1 to 20.

5. A method for manufacturing a modified compound, comprising:
mixing a first material having a polyether segment, a second material having a carboxyl group, and a solvent, wherein the first material has an end group, and the end group is a hydroxyl group, a mercapto group, an amino group or a carboxyl group, and the second material is formed by reacting amino acid or peptide with alkacrylic acid, acrylic acid, or acyl halide having an acryl group or an alkylacryl group, and the second material reacts with the end group of the first material through a carboxyl group to obtain the modified compound.

6. The method of claim 5, wherein the step of mixing the first material, the second material, and the solvent is carried out in an inert gas environment.

7. The method of claim 5, wherein the first material is polyether, and the second material is N-methacryloylglycine.

8. The method of claim 7, wherein the polyether is polyethylene glycol monomethyl ether.

9. The method of claim 5, wherein the first material is polyether, and the second material is formed by reacting the peptide with the acyl halide having a methacryl group.

10. The method of claim 9, wherein the polyether is poly(ethylene glycol).

11. The method of claim 5, wherein the first material is polyether, and the second material is formed by reacting glycine with methacrylic acid or the acyl halide having a methacryl group.

12. The method of claim 11, wherein the polyether is poly(ethylene glycol).

13. A method for manufacturing a modified compound, comprising:
mixing a material having a polyether segment, an amino acid derivative having a protecting group, a solvent, and acyl halide having an acryl group or an alkylacryl group, wherein the material has an end group, and the end group is a hydroxyl group, a mercapto group, an amino group or a carboxyl group, and the amino acid derivative reacts with the end group of the material through a carboxyl group, and the amino acid derivative reacts with the acyl halide through an amino group, to obtain the modified compound.

14. The method of claim 13, wherein the step of mixing the material having the polyether segment, the amino acid derivative having the protecting group, the solvent and the acyl halide comprises:
mixing the material, the amino acid derivative, and the solvent, allowing the amino acid derivative to react with the end group of the material through a carboxyl group to form a transition product, wherein the transition product has an amino group; and
mixing the transition product, the acyl halide, and the solvent, allowing the amino group of the transition product to react with the acyl halide to obtain the modified compound.

15. The method of claim 14, wherein the material is polyether, and the amino acid derivative is histidine derivative, and the acyl halide is methacryl halide.
